(19) United States
(12) Patent Application Publication
(10) Pub. No.: US 2019/0264255 A1
(43) Pub. Date: Aug. 29, 2019

(54) Competition Test of an Enzyme Substrate with Internal Compensation for Enzyme Activity

(71) Applicant: TECHNISCHE UNIVERSITÄT DARMSTADT, Darmstadt (DE)

(72) Inventors: Katja SCHMITZ, Gross-Zimmern (DE); Christian WEBER, Hotheim am Taunus (DE); Joachim HÖNES, Zwingenberg (DE); Bastian E. RAPP, (DE); Julia Susanna WACK, Pfungstadt (DE)

(21) Appl. No.: 16/349,033

(22) PCT Filed: Nov. 10, 2017
(86) PCT No.: PCT/EP2017/078897

§ 371 Date: May 10, 2019

(30) Foreign Application Priority Data
Nov. 10, 2016 (DE) 10 2016 121 553.8

(51) Int. Cl. C12Q 1/56

(52) U.S. Cl. CPC C12Q 1/56

(57) ABSTRACT

A method for determining the concentration of an analyte in a sample, and to a test strip and a kit for carrying out said method, and a test system having said test strip and a detector. The use of the method to determine the concentration of an analyte in a sample.

### FIELD OF THE INVENTION

The present invention relates to a method for determining the concentration of an analyte in a sample, as well as a test strip for carrying out the method, and a test system comprising the test strip and a detector. The invention further relates to the use of the method for determining the concentration of an analyte in a sample.

### BACKGROUND OF THE INVENTION

As the final factor in the coagulation cascade, fibrinogen is of central importance. Fibrinogen is cleaved by thrombin. This is the step in the blood clotting cascade that leads to fibrin monomers and, through their aggregation, to the formation of the first, mechanically still labile clot.

As a result of blood loss (e.g. from surgery or severe injury), blood thinning (e.g. by infusion therapy), or the consumption of coagulation factors (e.g. perioperatively during cardiac surgery or complex general surgery operations), a general coagulation factor deficiency or isolated fibrinogen deficit may develop. This can cause severe bleeding. Blood fibrinogen levels decline rapidly in many patients with severe trauma, and low levels of fibrinogen are associated with increased mortality rates. There are no fibrinogen reserves outside the blood plasma. Therefore, in the event of rapid decline, the fibrinogen level cannot be normalized sufficiently by the body's own mechanisms. The deficits must therefore be compensated for by external supply. In the past, fresh frozen plasma (FFP) was often used to correct a fibrinogen deficit. However, this always involves the risk of transmission of infection or the induction of kidney or multi-organ failure. In addition, when FFP is administered, a high volume (>15-20 ml/kg of body weight) must be transfused, further burdening the patient's already highly stressed cardiovascular system, which may have dramatic consequences. Therefore, increasingly, one does not transfuse the patient with FFP, but only supplements the substances that are really needed, in the form of clotting factor concentrates.

In order to be able to use fibrinogen in a targeted manner, in the course of an operation it is necessary to carry out within minutes an exact determination of the fibrinogen level. Various tests are known in the prior art for determining fibrinogen concentration, including the Clauss test, thromboelastometry (e.g. by ROTEM®), and gravimetric determination.

The standard fibrinogen test is the Clauss test. An excess of thrombin rapidly cleaves the fibrinogen. Fibrin is formed, which coagulates into a clot, resulting in turbidity of the sample, which may be detected. The time to achieve threshold turbidity is related to the fibrinogen concentration, so that this time may be used to determine the fibrinogen concentration. However, the blood matrix has an influence; both the turbidity intensity and the time to the threshold are affected. A higher specificity is therefore desirable. The test requires well-trained laboratory personnel and appropriate equipment. In routine use, approximately 55 minutes of processing time is to be expected. As a result, the results usually come too late, or no longer reflect the current situation. Depending on the bleeding dynamics, completely different fibrinogen concentrations may be present just 40 minutes after sampling the blood.

The ROTEM® test from TEM International may be performed in the operating theatre or in the intensive care unit. It is based on thromboelastometry. Results are obtained in about 10 minutes. However, at the intervention threshold of about 1 g/L the test is at the lower limit of detection and is therefore inaccurate. It is also heavily influenced by surgical measures such as infusion of plasma expanders. In addition, it should be noted that devices for performing thromboelastometry are very expensive and therefore such devices are usually found only in university clinics and other extremely large hospitals.

Gravimetry is complex to carry out and requires much more time than the Clauss test.

A rapid test with high accuracy also in the lower concentration range (<1.5 g/L) is currently not available. However, this concentration range is of particular importance for reduced fibrinogen levels. The current guideline on coagulation management in bleeding patients after trauma (Rossaint et al., Critical Care (2016) 20:100, Recommendation 28 on pages 24 and 25) therefore recommends carrying out a “blind” initial treatment using 3 to 4 g of fibrinogen concentrate, further fibrinogen therapy being adjusted on the basis of the subsequently determined fibrinogen concentrations. Similar recommendations apply in, amongst other, gynecology (peripartum hemorrhage), cardiac surgery (bleeding during surgery on heart-lung machines), transplantation surgery (especially liver transplantation), and also “internal medicine” (gastric ulcer bleeding). Due to the lack of alternative test methods for the determination of the fibrinogen concentration, current practice therefore necessarily involves acceptance of the fact that, due to the “blind” initial therapy, during fibrinogen administration overdosing or underdosing may occur, either of which may lead to fatal effects.

There is therefore an urgent need for a rapid test for determining fibrinogen concentration with high accuracy, especially in the lower concentration range (<1.5 g/L).

Known fibrinogen tests are based on a test principle involving fibrin monomer being rapidly produced by a thrombin excess, the polymerization of which then generates a signal. The measured signal may relate to turbidity of the sample (Clauss test), an increase in the viscosity of the sample (thromboelastometry), or gravimetrically determinable clot formation. For the reasons given above, however, none of the known test methods are used in practice, since the Clauss test and gravimetry take too much time, while thromboelastometry offers insufficient accuracy at the relevant fibrinogen concentrations in the range <1.5 g/L and the readings are affected by other parameters such as factor XIII or infused colloidal infusion solution.

### SUMMARY OF THE INVENTION

In accordance with the present invention, these problems have been overcome. The invention is based on the use of a test principle that is completely different to the known methods. This principle is illustrated in the present description using the example of determining fibrinogen concentration, but is generally applicable to enzyme-kinetic determinations of substrate concentrations.

The solution according to the invention involves determination of the fibrinogen concentration via an enzyme-kinetic method. Fibrinogen and a signal-producing, in particular chromogenic or fluorogenic, substrate are cleaved by thrombin. The generation of an electrochemical signal is also possible. As a result of the conversion of the signal-producing substrate by the enzyme, a signaling agent, in particular a dye, fluorophore or reducing agent, is produced, the formation of which is detected. Detection takes place, for example, by measuring the absorption or the fluorescence at a specific wavelength or via the current flow at a suitably selected potential.

Fibrinogen and the signal-producing substrate compete for conversion by the enzyme. The more fibrinogen is present in the analysis solution, the less chromogenic substrate is cleaved, i.e. the lower the increase in absorption. This competition is known from the literature (for example Mathur, Biochemistry 1993, 32, 7568).

Such an enzymatic competition test depends directly on the enzyme activity and, by virtue of the latter's temperature dependence, on the test temperature. In addition, the activity of the enzyme usually decreases during storage of the test. Depending on the time and conditions of storage, the residual activity is variable. Also, the pH and the measurement wavelength are factors that strongly influence the test result. One would therefore have to keep all these conditions very precisely constant in order to obtain a reliable result. Since this is hardly possible, especially for the enzyme activity, one would instead have to carry out calibration with a known fibrinogen concentration before the actual determination. However, fibrinogen is also not stable. Above all, however, the calibration solution would differ greatly from the blood matrix, which in turn influences the thrombin activity. As a result, the calibration would be highly liable to error. Also, the extra step would make handling more complex and slower.

Because of these difficulties, methods known from the prior art for determining fibrinogen concentration are not based on such an enzyme-kinetic competition test. The inventors of the present invention have nonetheless found that a reliable concentration determination via such a competition test is possible even without complex calibration, when two different signals produced by the activity of the enzyme are detected and then offset against each other, in order to eliminate the dependence on difficult-to-control factors such as temperature, pH and enzyme activity. Theoretically, the same applies to the amount of active enzyme. For temperature and pH, small residual disturbances may be possible through change of the K_{M} values. However, it is to be expected that these residual disturbances will be extremely low, especially if the signal-producing substrate and analyte have a similar structure. Also, additional enzyme or inhibitors/activators from the sample are compensated for because they act equally on both measurements. The decisive advantage of the principle according to the invention is therefore the simple compensation for disturbances of all kinds. Fundamentally, there are various possible ways to implement this inventive principle.

If not only the enzymatic conversion of the signal-producing substrate, but also the enzymatic conversion of the analyte produces a measurable signal, there is a possibility of implementing the inventive principle by producing the two signals in parallel, e.g. at several wavelengths, in order to detect and properly offset the measured values. Both reactions depend in the same way on enzyme activity and temperature as well as other difficult-to-control factors, since both reactions take place in one and the same analysis solution. The conversion of the signal-producing substrate then serves as an internal standard. Therefore, if the measured values are suitably correlated with one another, the dependence on these influencing factors may be eliminated to such an extent that errors due to influencing factors that are difficult to control may be avoided.

A measurable signal, though, is not produced in all cases when the analyte is converted by the enzyme. However, since the principle according to the invention is based on the detection and offsetting of at least two different signals produced by the activity of the enzyme, in embodiments of the invention in which the conversion of the analyte does not provide a detectable signal, the two signals must be produced by conversion of the signal-producing substrate. There exist at least the following two possibilities for this.

On the one hand, the signals may be produced in one and the same analysis solution. This requires, however, that the two signals are distinguishable. One way to do this is to use two different signal-producing substrates which, when converted by the enzyme, give distinguishable signals. For example, it is conceivable that upon conversion of a first signal-producing substrate by the enzyme, a first dye is formed and that, upon conversion of a second signal-producing substrate by the enzyme, a second dye is formed, wherein the first and the second dyes are distinguished, for example, by the wavelengths of their absorption maxima. Again, both reactions depend in the same way on enzyme activity and temperature as well as other, difficult-to-control influencing factors, since both reactions take place in one and the same analysis solution. Therefore, for these embodiments of the invention too, if the measured values are suitably correlated with each other, the dependence on such influencing factors may be eliminated to the extent that errors due to influencing factors that are difficult to control may be avoided.

On the other hand, the principle according to the invention may be implemented by carrying out two parallel enzymatic reactions at different concentrations of the signal-producing substrate. Preferably, in such an embodiment, one reaction is performed at a very high concentration of the signal-producing substrate. As a result, the measured activity of the enzyme is virtually independent of the presence of the analyte. With the aid of this reaction, therefore, the maximum initial rate vₘₐₓ of substrate conversion may be determined. The other reaction is preferably carried out at a much lower concentration of the signal-producing substrate. In this case, more analyte occupies the binding sites of the enzyme and accordingly less signal-producing substrate is converted. In both cases, the amount of converted signal-producing substrate is equally dependent on enzyme activity and temperature. If one divides the determined initial rates into each other, the dependencies on the enzyme activity and the temperature and other influencing factors are eliminated. This also applies to different sensitivities of the detection reaction, e.g. in the case of substances which, for example, influence/quench the fluorescence of the product. These embodiments, too, allow a rapid and reliable determination of fibrinogen concentration with high accuracy even in the lower concentration range (<1.5 g/L) without the need for prior calibration.

The problems of the prior art are solved by the subject-matter of the claims. The problems are solved, in particular, by a method for determining the level of an analyte in a sample comprising the following steps:

a. Providing at least one analysis solution,
i. wherein the at least one analysis solution comprises in each case an enzyme, a signal-producing substrate and a known proportion of the sample to be analyzed, and
ii. wherein the enzyme is capable of converting both the analyte and the signal-producing substrate so that the analyte and signal-producing substrate compete for conversion by the enzyme,

b. Detection of two signals S1 and S2 produced by enzyme-catalyzed conversion in the at least one analysis solution,

c. Calculation of a conversion factor from the signals, and

d. Determination of the content of the analyte in the sample by means of the conversion factor.

### DETAILED DESCRIPTION OF THE INVENTION

The method according to the invention serves to determine the level of an analyte in a sample. In principle, all substances which may be converted enzymatically are suitable as the analyte. Preferably, the analyte is a peptide, especially a polypeptide. Most preferably, the analyte is a protein. Both monomeric and oligomeric proteins are suitable as analytes according to the invention. In a particularly preferred embodiment, the analyte is fibrinogen.

Optionally, prior to the step of providing the at least one analysis solution, the method according to the invention may comprise the step of providing the sample to be analyzed. However, the step of providing the sample to be analyzed is preferably not part of the method according to the invention. Rather, the method may be used to analyses an independently-provided sample. This sample is the sample to be analyzed for the level of the analyte. In principle, samples of any kind may be considered. Preferably, the sample is an aqueous sample. According to the invention, an aqueous sample is a sample having a water content of at least 25% by weight, more preferably at least 40% by weight, even more preferably at least 45% by weight, even more preferably at least 49.5% by weight. Preferably, the sample is selected from the group consisting of blood, urine, saliva, milk and sweat. In preferred embodiments, the sample is a blood sample. However, the method according to the invention may also be used for the analysis of laboratory solutions, food extracts or water samples, in particular drinking water samples, for the content of a particular enzymatically-convertible analyte.

The sample may be processed prior to providing the analysis solution. For example, the sample may be diluted if it is to be expected that the concentration of the analyte is particularly high. This dilution factor must be taken into account when determining the content of the analyte in the original sample. Preferably, the concentration of the analyte in the sample used according to step a of the method is in a range from 0.1*Kᵢ to 100*Kᵢ, preferably from 0.2*Kᵢ to 20*Kᵢ, even more preferably from 0.5*Kᵢ to 10*Kᵢ, where K is the Michaelis constant described below (Equations 1-4) for the conversion of the analyte by the enzyme that competes with the conversion of the signal-producing substrate. For the concentrations mentioned, particularly robust results are achieved with the method according to the invention. The exact concentration of the analyte in the sample is not known before carrying out the method according to the invention. This is first determined by the method. However, a concentration range in which the concentration of the analyte will lie with a high probability will usually be known to the person skilled in the art, so that a rough estimate in advance is possible. Depending on the molecular weight of the analyte, the preferred weight concentration may assume different values. For preferred analytes, especially fibrinogen, the concentration of the analyte in the provided sample is preferably in a range from 0.05 to 20 mg/ml, more preferably from 0.1 to 10 mg/ml, more preferably from 0.2 to 5 mg/ml, more preferably from 0.5 to 4 mg/ml.

Furthermore, for example, the pH of a sample may be adjusted if the originally provided sample has a pH that could inhibit enzyme activity in the analysis solutions. Preferably, the pH of the sample used according to step a of the method is in a range from 6 to 9, more preferably from 6.5 to 8.5, more preferably from 7 to 8. More preferably, the pH of the sample used in accordance with step a lies in a range of ±1.5, more preferably ±1.0, even more preferably ±0.5 of the optimum pH of the enzyme used. In the context of the invention, the optimum pH of the enzyme used is the pH at which the enzyme has the highest activity.

The provided sample is preferably a liquid sample, in particular an aqueous sample. However, solid samples are also conceivable, for example stool samples, soil or stone samples or samples in powder form. Soluble solid samples may preferably be used directly to provide the analysis solution. Alternatively, the analyte may be extracted from a solid sample and the recovered extract used as a proportion of the sample to provide the analysis solution. In such embodiments, the concentration of the analyte in the extract may be used to determine the level of the analyte in the solid sample.

According to step a of the method of the invention, at least one analysis solution comprising an enzyme, a signal-producing substrate and a known amount of the sample is provided. The enzyme may convert both the analyte and the signal-producing substrate so that the analyte and signal-producing substrate compete for conversion by the enzyme. In other words, the signal-producing substrate competes with the conversion of the analyte by the enzyme and the analyte competes with the conversion of the signal-producing substrate by the enzyme. The analyte and the signal-producing substrate thus behave as competitors.

The at least one analysis solution of the invention is an aqueous solution which allows the conversion of the analyte and the signal-producing substrate by the enzyme. To this end, the analysis solution may be adapted in terms of its composition and temperature to the requirements of the respective enzyme. In particular, the pH and/or the salt concentration may be adjusted according to the requirements of the respective enzyme. According to the invention, an aqueous solution is a solution having a water content of at least 50% by weight, more preferably at least 75% by weight, more preferably at least 90% by weight, even more preferably at least 95% by weight.

According to step a of the method of the invention at least one analysis solution is provided. In preferred embodiments of the invention, exactly one analysis solution is provided. In other preferred embodiments, exactly two analysis solutions are provided. In particularly preferred embodiments, a first analysis solution A1 and a second analysis solution A2 are provided, wherein the first analysis solution A1 and the second analysis solution A2 each comprise an enzyme, a signal-producing substrate and a known amount of the sample, and wherein the concentration of the signal-producing substrate in the second analysis solution A2 is significantly lower than, preferably at most half as high as, the concentration of the signal-producing substrate in the first analysis solution A1. Such embodiments are particularly advantageous if the enzymatic conversion of the analyte provides no detectable signal and no different signal-producing substrates are present which could give distinguishable signals upon enzymatic conversion. In such embodiments, the signals S1 and S2 produced by enzymatic conversion of the signal-producing substrate are not distinguishable from each other as such, so detection must occur in two separate analysis solutions. Preferably, the different analysis solutions contain the same amount of enzyme. As a result, an additional standardization step may be avoided. In addition, in this way, any influence of the enzyme concentration on parameters to be regarded as constant between the reactions is excluded. Preferably, the various analysis solutions contain the same amount of the sample. As a result, an additional standardization step can be avoided. In addition, in this way any influence of the concentration of the analyte on parameters to be considered as constant between the reactions is excluded. The term “same amount” means, in the context of the invention, that the deviations are not more than ±1%.

According to step a of the method of the invention, the at least one analysis solution comprises an enzyme. According to the invention, all enzymes which can enzymatically convert the analyte and the signal-producing substrate are suitable, wherein a detectable signal is produced at least by conversion of the signal-producing substrate. Preferably, the enzyme is selected from the group consisting of protein-based enzymes, ribozymes and deoxyribozymes. Most preferably, the enzyme is a protein. More preferably, the enzyme is selected from the group consisting of oxidoreductases, transferases, hydrolases, lyases, isomerases and ligases. More preferably, the enzyme is selected from the group consisting of hydrolases and transferases. In the case of transferases, the principle of the invention may be applied, provided that a suitable signal-producing substrate is present, in which the transfer of a chemical functionality leads to a measurable signal, in particular a change of absorption or fluorescence, and which can compete with the analyte. In the case of hydrolases, the signal is preferably produced by hydrolytic cleavage of a signaling agent from the signal-producing substrate. Most preferably, the enzyme is a hydrolase. Even more preferably, the enzyme is a peptidase. Even more preferably, the enzyme is a serine protease. Even more preferably, the enzyme is selected from the group consisting of thrombin and trypsin. Even more preferably, the enzyme is thrombin.

According to the invention, the at least one analysis solution comprises at least one signal-producing substrate. The signal-producing substrate may be any substrate that can be converted by the enzyme, and by the conversion of which a detectable signal is produced. Preferably, a signaling agent is formed by the enzymatic conversion of the signal-producing substrate. The signaling agent is preferably a dye. For the purposes of this invention, a dye is to be understood as meaning a substance which may be detected optically by means of spectrometers or photometers. Dyes in the context of this invention are, for example, substances which absorb light of a specific wavelength, so that the concentration of the substances may be determined by the absorption of the analysis solution at this wavelength. However, dyes within the meaning of this invention may also be fluorophores, i.e. fluorescent components which not only absorb light of a certain wavelength but also emit light of a wavelength higher than the absorption wavelength, so that the concentration of these dyes may be detected by the amount of emitted light. Fluorescent dyes may be produced enzymatically, in particular by transferases, hydrolases, lyases or isomerases.

In other preferred embodiments, the signal-producing substrate contains two molecular units that form a FRET (Förster Resonance Energy Transfer) pair as donor and acceptor. Preferably, donor and acceptor are positioned sufficiently close to each other to enable FRET with high efficiency. The donor is thereby a fluorophore. The acceptor is a dye that can absorb light in the wavelength range of emission of the donor. Upon excitation of the donor with light of appropriate wavelength, the energy absorbed is transferred to the acceptor so that the fluorescence of the donor is greatly reduced. The acceptor may be a fluorescent dye. In this case, upon excitation of the donor, the acceptor may emit light at the characteristic acceptor emission wavelengths. If the acceptor is not a fluorescent dye, no fluorescence is observed. By enzymatic cleavage of the substrate, the donor and acceptor are separated from each other, whereby the strong distance-dependent energy transfer between the donor and acceptor is inhibited and there is an increase in the emission of the donor.

However, the signaling agent does not necessarily have to be a dye. In particular, electrochemically detectable signaling agents are also in accordance with the invention. In embodiments in which the signaling agent is detectable electrochemically, signal-producing substrates are preferably used which contain substrate-bound, particularly preferably peptide-bound, p-aminophenol and/or p-phenylenediamine residues. At a suitable potential, the substrate-bound residue cannot be oxidized, whereas the cleaved free amine may be well oxidized. Such signal-producing substrates are therefore particularly suitable for use with hydrolases. Further preferred is the use of these electrochemical signal-producing substrates with peptidases.

As described above, peptidases are particularly preferred enzymes for the purposes of the present invention. Particularly preferred signal-producing substrates according to the present invention are therefore those in which the signal is produced by cleavage of a peptide bond. Particularly preferred are those signal-producing substrates in which the cleavage of a peptide bond leads to the formation of a signaling agent, in particular a dye as that term in used in the context of the invention. Particularly preferably, the signal-producing substrate comprises a p-nitroaniline group which is connected via its amino group to the carboxyl group of an amino acid, more preferably to the carboxyl group of a basic amino acid, most preferably to the carboxyl group of arginine, via a peptide bond. When this peptide bond is cleaved by the enzyme, p-nitroaniline is released. The concentration of p-nitroaniline may be detected spectroscopically at a wavelength of 405 nm. P-nitroaniline is a particularly preferred dye according to the present invention. Particularly preferred signal-producing substrates according to the present invention are N-benzoyl-D,L-arginine-p-nitroanilide (BAPNA) and the oligopeptide substrates Ala-Gly-Arg-p-nitroanilide and p-tosyl-Gly-Pro-Arg-p-nitroanilide. By choosing other peptides, virtually all known peptidases and their natural substrates may be selected, so that the test principle is transferable to many applications.

Particularly preferred are those signal-producing substrates in which the cleavage of a peptide bond leads to the formation of a signaling agent, in particular a fluorescent dye as that term in used in the context of the invention. Particularly preferably, the signal-producing substrate comprises a coumarin group, in particular a 7-amino-4-methylcourmarin group, connected via its amino group with the carboxyl group of an amino acid, more preferably with the carboxyl group of a basic amino acid, most preferably with the carboxyl group of arginine, via a peptide bond. When this peptide bond is cleaved by the enzyme, 7-amino-4-methylcourmarin is released. The concentration of 7-amino-4-methylcourmarin may be detected spectroscopically by absorption at a wavelength of about 340 nm and by emission at a wavelength of about 460 nm.

Also in accordance with the invention are embodiments in which the signal-producing substrate itself is a signaling agent as that term in used in the context of the present invention, wherein this signaling agent is broken down by the enzymatic conversion. In such embodiments, the detected signal shows a decrease in absorbance or a decrease in fluorescence.

Also in accordance with the invention are embodiments according to which not only the signal-producing substrate but also the substance formed by enzymatic conversion of the signal-producing substrate are signaling agents, if their signals can be distinguished from each other. Frequently, for example, a signal-producing substrate will absorb electromagnetic radiation of a certain wavelength, while a signaling agent formed by enzymatic conversion of the signal-producing substrate absorbs radiation of another, in particular higher, wavelength. Such pairs of signal-producing substrate and signaling agent formed therefrom are also in accordance with the invention.

According to the invention, the at least one analysis solution also comprises a known proportion of the sample. The principle according to the invention involves determining the content of the analyte in the sample by detecting at least two signals produced by enzyme-catalyzed conversion and their subsequent offsetting. In order to be able to draw from the signals produced in the at least one analysis solution conclusions about the content of the analyte in the sample, it is necessary to know what proportion of the sample has found its way into the analysis solution. The proportion of the sample contained in the analysis solution must therefore be known for a successful implementation of the method according to the invention.

According to step b of the method according to the invention, two signals S1 and S2 produced by enzyme-catalyzed conversion in the at least one analysis solution are detected. The nature of the signals depends on the signal-producing substrate used. The signal is preferably an optically detectable signal, in particular a change in the absorption in the analysis solution or a fluorescence signal. A change in the absorption may, in particular, consist of a change in the extinction coefficient and/or a change in the absorption maximum. In particular, a fluorescence signal may consist of a change in the quantum yield and/or a change in the excitation and/or emission wavelength.

A detectable signal in the sense of the present invention does not necessarily have to be an optically detectable signal. According to the invention, signals are also detectable in other ways, in particular electrochemically detectable signals.

The signals are preferably produced by (i) enzyme-catalyzed conversion of the signal-producing substrate (signal S1) and by enzyme-catalyzed conversion of the analyte (signal S2), or (ii) by enzyme-catalyzed conversion of a higher concentration of the signal-producing substrate (signal S1) and by enzyme-catalyzed conversion of a lower concentration of the signal-producing substrate (signal S2), or (iii) by enzyme-catalyzed conversion of a first signal-producing substrate (signal S1) and by enzyme-catalyzed conversion of a second signal-producing substrate (signal S2). In these embodiments, the two signals S1 and S2 are thus produced under comparable or even identical conditions in relation to difficult-to-control parameters such as temperature or enzyme activity. By the detection of two signals and their subsequent offsetting in accordance with the present invention, it is possible to eliminate the influence of such difficult-to-control factors, thereby enabling rapid and reliable determination of the concentration of the analyte with high accuracy without the need for prior calibration.

According to preferred embodiments, a signal S1 is produced by enzyme-catalyzed conversion of the signal-producing substrate and a signal S2 by enzyme-catalyzed conversion of the analyte. In so far as the signals S1 and S2 in such embodiments can be distinguished from one another, which is frequently the case because of the differing signal-producing molecules (signal-producing substrate on the one hand and analyte on the other hand), the signals may be detected in one and the same analysis solution, so that the method may be successfully implemented with a single analysis solution.

According to further preferred embodiments, a signal S1 is produced by enzyme-catalyzed conversion of a higher concentration of the signal-producing substrate and a signal S2 by enzyme-catalyzed conversion of a lower concentration of the signal-producing substrate. In such embodiments, the two signals S1 and S2 cannot be distinguished per se because they are based on the same signal-producing molecules. Therefore, in such embodiments, two separate analysis solutions are provided to allow differentiation of the signals S1 and S2.

In such embodiments, therefore, a first analysis solution A1 and a second analysis solution A2 are provided, wherein the signal S1 is produced in the first analysis solution A1 and the second signal S2 in the second analysis solution A2. The terms first and second analysis solutions are not intended to indicate that the analyses must be performed sequentially or even in a particular order. Rather, the two analyses are preferably carried out in parallel. The first analysis solution A1 and the second analysis solution A2 each include an enzyme, a signal-producing substrate and a known amount of the sample. The concentration of the signal-producing substrate in the first analysis solution A1 is higher than the concentration of the signal-producing substrate in the second analysis solution A2. Preferably, the concentration of the signal-producing substrate in the first analysis solution A1 is at least twice, more preferably at least three times, more preferably at least five times, even more preferably at least ten times, more preferably at least twenty times as high, even more preferably at least fifty times as high, as the concentration of the signal-producing substrate in the second analysis solution A2. A large difference in concentration of the signal-producing substrate in the analysis solutions contributes to a robust and low-error determination of the concentration of the analyte.

The concentration of the signal-producing substrate in the first analysis solution A1 is preferably at least 80%, more preferably at least 90%, more preferably at least 95%, more preferably at least 99%, even more preferably at least 99.9%, of the concentration required to saturate the enzyme. The higher the concentration of the signal-producing substrate, the more robust the measured signal. In this context, the concentration required for saturation of the enzyme is to be understood as meaning the concentration of the signal-producing substrate in the first analysis solution A1 from which an increase of the concentration of the signal-producing substrate by 10% is accompanied by an increase of the signal of not more than 0.1%.

The concentration of the signal-producing substrate in the first analysis solution A1 is therefore preferably comparatively high. Preferably, the concentration of the signal-producing substrate in the first analysis solution A1 is at least 10*K_{M}, more preferably at least 25*K_{M}, more preferably at least 50*K_{M}, more preferably at least 100*K_{M}, even more preferably at least 200*K_{M}, where K_{M} is the Michaelis constant described below (Equations 1 to 4).

The concentration of the signal-producing substrate in the second analysis solution A2 may be varied within a relatively wide range. With very low values, one obtains very high conversion factors, but must take into account that the conversion rate is very low and correspondingly difficult to measure. Preferably, therefore, the concentration of the signal-producing substrate in the second analysis solution A2 is in a range from 1*K_{M} to 20*K_{M}, more preferably from 2*K_{M} to 10*K_{M}, most preferably from 4*K_{M} to 8*K_{M}, where K_{M} is the Michaelis constant described below (Equations 1 to 4).

According to further preferred embodiments, a signal S1 is produced by enzyme-catalyzed conversion of a first signal-producing substrate and a second signal S2 by enzyme-catalyzed conversion of a second signal-producing substrate. These embodiments are similar to the embodiments described above in which the signal S1 is produced by enzyme-catalyzed conversion of the signal-producing substrate and the signal S2 by enzyme-catalyzed conversion of the analyte. However, according to the embodiments described here, in contrast to the embodiments described above, the second signal is produced not by conversion of the analyte but by conversion of a second signal-producing substrate. In these embodiments, the analysis solution thus contains both the analyte and two different signal-producing substrates. The method according to such embodiments may usually be carried out in a single analysis solution, provided that the signals S1 and S2 can be distinguished from one another. Such embodiments therefore offer the possibility of implementing the method in a single analysis solution, even if the analyte does not give a detectable signal upon enzymatic conversion. A prerequisite for these embodiments, however, is that there are two different signal-producing substrates that lead to distinguishable signals upon enzymatic conversion.

In particularly preferred embodiments, neither the signal S1 nor the signal S2 are produced by enzyme-catalyzed conversion of the analyte. In other words, it is particularly preferred if both the signal S1 and the signal S2 are produced by enzyme-catalyzed conversion of at least one signal-producing substrate, wherein the analyte is not a signal-producing substrate.

According to step c of the method according to the invention, a conversion factor is calculated from the detected signals. The conversion factor is preferably calculated from the signals by determining from the signals the initial rates v₀(S1) and v₀(S2) of the enzymatic conversion of the signal-producing substrate and these initial rates v₀(S1) and v₀(S2) are offset with each other. Particularly preferably, the conversion factor is calculated as the quotient v₀(S2)/v₀(S1) or as its reciprocal v₀(S1)/v₀(S2).

The initial rate v₀ of the enzymatic conversion of the signal-producing substrate is given for the simplest case of competitive inhibition as:

v₀=vₘₐₓ[S]/(1+[I]/Kᵢ)K_{M}+[S]. (Equation 1)

Here, vₘₐₓ is the maximum conversion rate, which corresponds to the product of the turnover number k_{cat} and the concentration of the active enzyme [E]₀. [S] is the concentration of the signal-producing substrate. K_{M} is the Michaelis constant of the conversion of the signal-producing substrate by the enzyme. K is the Michaelis constant of the conversion of the analyte by the enzyme. Since the analyte acts as an inhibitor of the conversion of the signal-producing substrate, K is also referred to as an inhibitor constant in the context of the invention. [I] is the concentration of the analyte to be determined. The terms “Kᵢ” and “K_{I}” are used synonymously in the present specification.

The initial rate v₀ may be determined from the measured signal. This is well known to those skilled in the art. Since it is known how much signal-producing substrate was used, the concentration [S] is also known. K_{M} and Kᵢ are experimentally determinable quantities to be established by calibration experiments with signal-producing substrate and analyte at series of varying concentrations. Nevertheless, the concentration [I] of the analyte cannot readily be determined from the initial rate v₀, since the maximum conversion rate vₘₐₓ of the signal-producing substrate is not known. As described above, for vₘₐₓ, unlike other parameters such as K_{M} and Kᵢ, appropriate calibration experiments carried out in advance do not help, because vₘₐₓ is very much dependent on difficult-to-control factors such as temperature, enzyme activity and properties of the complex sample matrix. Therefore, according to the invention, a conversion factor is calculated from the detected signals. This conversion factor is largely independent of the difficult-to-control influencing factors, since these factors influence the measurement of the first signal S1 and the measurement of the second signal S2 in the same way, and may therefore be eliminated by offsetting the signals to give a conversion factor.

A particularly simple calculation of the conversion factor is then possible if in a reaction very much signal-producing substrate and very little analyte is present, so that the initial rate v₀ of the conversion of the signal-producing substrate corresponds to the maximum conversion rate vₘₐₓ (e.g. v₀(S1)=vₘₐₓ). If the concentrations of enzyme and analyte in the first analysis solution A1 are equal to the concentrations of enzyme and analyte in the second analysis solution A2, the conversion factor U in such a case is:

U=v₀(S2)/v₀(S1)=[S]/(1+[I]/Kᵢ)K_{M}+[S] (Equation 2)

Of course one may also use the reciprocal 1/U instead of U.

In general, the formula for the conversion factor is:

U=v₀(S2)/v₀(S1)=[S₂]vₘₐₓ/K_{M}+Kₘ[I]/Kᵢ+[S₂]*K_{M}+Kₘ[I]/Kᵢ+[S₁]/[S₁]vₘₐₓ=[S₂]/[S₁]*K_{M}+Kₘ[I]/Kᵢ+[S₁]/K_{M}+Kₘ[I]/Kᵢ+[S₂] (Equation 3)

Without analyte ([I]=0), U assumes the value U(0)=[S₂]/[S₁]*K_{M}+[S₁]/K_{M}+[S₂]. For large quantities of analyte, U approaches the limit [S2]/[S1]. The dynamic width is thus determined by the concentration ratio of the first and second signal-producing substrates. The smaller the difference in concentration, the smaller is the dynamic width and the flatter the slope. The general curve of U([I]) is shown in FIG. 1. The reciprocal yields the inverse curve with positive slope.

For small analyte concentrations [I], the function U([I]) is approximately linear and may be approximated by the first two terms of the Taylor series:

Ũ([I])=[S₂]/[S₁]*K_{M}+[S₁]/K_{M}+[S₂]+[S₂]*K_{M}/[S₁]*Kᵢ*[S₂]−[S₁]/(K_{M}+[S₂])²[I] (Equation 4)

The slope is steepest for [I] in the region of K and the determination of the analyte concentration is possible with the greatest accuracy in this region. The determination of U by forming a quotient of the two initial rates v₀(S1) and v₀(S2) may thus be used to reliably determine the concentration of the analyte even if v₀(S1) is clearly removed from vₘₐₓ. Preferably, however, v₀(S1) is at least 0.5*vₘₐₓ, more preferably at least 0.8*vₘₐₓ.

Therefore, the concentrations of enzyme and analyte in the first analysis solution A1 are preferably equal to the concentrations of enzyme and analyte in the second analysis solution A2, in order to allow the simplest possible calculation of the conversion factor. In embodiments in which the concentrations of enzyme and analyte in the first analysis solution A1 are not equal to the concentrations of enzyme and analyte in the second analysis solution A2, further normalization factors must be introduced into the above equation. This is, however, a straightforward calculation for the person skilled in the art. However, account must also be taken of the fact that with the use of a different amount of analyte and therefore also sample quantity, interfering sample constituents are present in different concentrations in analysis solutions 1 and 2. Such an embodiment is therefore not preferred.

According to step d of the method according to the invention, the level of the analyte in the sample is determined by means of the conversion factor. In the Equations 2 to 4 given above for determining the conversion factor U as a quotient of v₀(S2) and v₀(S1), the parameter vₘₐₓ was eliminated in comparison to the above-mentioned Equation 1 for determining the initial rate v₀. Since the conversion factor U is calculated from the detected signals and the parameters [S], K_{M} and K are known as described above, the concentration [I] of the analyte in the analysis solutions and, since the analysis solutions contain a known proportion of the sample, also the content of the analyte in the sample may be determined using the calculated conversion factor.

According to particularly preferred embodiments, the determination of the concentration of the analyte is based on an empirical calibration curve. Such a calibration curve is preferably obtained by determining the conversion factor at different concentrations of the analyte. In this way, the dependence of the conversion factor on the concentration of the analyte may be determined, so that from the conversion factor, which is obtained for an unknown sample, the concentration of the analyte in this sample may be determined.

Particularly robust results are obtained for [I]≥Kᵢ.

Preferably, the concentration [I] of the analyte in the analysis solutions is at least 1*Kᵢ, more preferably at least 2*Kᵢ, more preferably at least 5*Kᵢ, more preferably at least 10*Kᵢ. However, the concentration [I] of the analyte in the analysis solutions should also not be too large, since otherwise the competition with the signal-producing substrate becomes very great, resulting in a lower signal strength. Preferably, the concentration [I] of the analyte in the analysis solutions is at most 1000*Kᵢ, more preferably at most 500*Kᵢ, even more preferably at most 200*Kᵢ. Kᵢ is the inhibitor constant described above.

Preferably, the ratio of the quotient [S]/K_{M} to the quotient [I]/Kᵢ in the second analysis solution A2 is in a range of 0.1 to 10, more preferably 0.2 to 5, further preferably 0.5 to 2, more preferably 0.8 to 1.2. Most preferably, the ratio of the quotient [S]/K_{M} to the quotient [I]/Kᵢ in the first analysis solution is about 1:1. In other words, particularly preferably the concentrations of analyte and signal-producing substrate in the second analysis solution A2 have the same relationship as K to K_{M}.

In contrast, in the first analysis solution A1 the ratio of the quotient [S]/K_{M} to the quotient [I]/Kᵢ is in a range from 10 to 1000, more preferably from 20 to 500, more preferably from 50 to 200, even more preferably from 80 to 120.

Thus, the method of the present invention enables rapid and reliable determination of the analyte concentration with high accuracy without the need for prior calibration.

The method of the invention may be carried out with standard laboratory equipment. Preferably, the analysis solutions are provided in cuvettes or in microtiter plates. In particular, the measurement is carried out in these or similar containers. This simplifies the detection of the signals produced, if they are optically detectable signals.

However, the method according to the invention may also be carried out with a test strip according to the invention. This is particularly advantageous because the method may then be easily implemented by even less experienced personnel and even directly at the site of the surgery, in the emergency department, in the operating theatre, delivery room or intensive care unit, for example to determine the fibrinogen concentration.

A test strip according to the present invention has a multilayer construction. Among other things, enzyme and substrates must be present separately. The test strip of the invention has at least two layers. The test strip preferably has at least three, more preferably at least four, more preferably at least five, even more preferably at least six layers. However, the test strip preferably has at most ten, more preferably at most eight layers. Otherwise, the construction becomes very complex and prone to error.

The test strip according to the invention comprises at least one enzyme layer, which contains the enzyme, and at least one substrate layer, which contains the signal-producing substrate. The enzyme and the signal-producing substrate do not come into contact with each other, so that there is no conversion of the signal-producing substrate by the enzyme. However, when an aqueous sample is applied to the test strip, it diffuses through the enzyme layer and the substrate layer and dissolves the enzyme and signal-producing substrate immobilized in the separated layers of the test strip, which thereby come into contact, so that an analysis solution according to the present invention is formed. The signal produced in the analysis solution may then be observed through at least one opening, which may optionally be covered with a transparent protective layer.

Depending on the embodiment, the test strip according to the invention may have one or more than one, preferably two, substrate layers. If the enzymatic conversion of the analyte itself leads to a detectable signal, the signal produced by enzyme-catalyzed conversion of the signal-producing substrate and the enzyme-catalyzed conversion of the analyte signal can, as described above, be used to calculate the conversion factor and thus to determine the level of the analyte in the sample. Provided that these signals are distinguishable from each other, the enzymatic conversion of the signal-producing substrate and the analyte may take place in one and the same analysis solution. In such embodiments, therefore, one substrate layer is sufficient. The same applies to embodiments in which two different signal-producing substrates are used, which lead to mutually distinguishable signals when converted by the enzyme. In such embodiments as well, the conversion of the two substrates may be carried out in one and the same analysis solution, so that one substrate layer is sufficient.

In other preferred embodiments, in particular if the enzymatic conversion of the analyte itself does not lead to a detectable signal and if only one signal-producing substrate is available, two separately readable substrate layers are required because the two signals to be detected cannot be distinguished from one another. In such embodiments, the test strip according to the invention therefore has at least two substrate layers which are readable separately from one another, preferably exactly two substrate layers which are readable separately from one another. The substrate layers preferably contain different amounts of signal-producing substrate. More preferably, one substrate layer contains a high amount of signal-producing substrate, while the other substrate layer contains a small amount of signal-producing substrate. Particularly preferably, the amount of the signal-producing substrate in the one substrate layer is at least twice, more preferably at least three times, more preferably at least five times, even more preferably at least ten times, more preferably at least twenty times as high, even more preferably at least fifty times as high, as the amount of signal-producing substrate in the other substrate layer. A large difference in the amount of the signal-producing substrate in the various substrate layers contributes to a robust and low-error determination of the concentration of the analyte.

It is important that the two substrate layers are separated so that mixing does not occur even when the sample diffuses through the layers. The various substrate layers are therefore preferably arranged next to one another in the test strip, whereas the arrangement of enzyme layer and substrate layer takes place “in series”, i.e. one above the other or one after the other, so that mixing of substrate and enzyme by the diffusing sample is ensured.

The test strip according to the invention preferably contains at most two enzyme layers, more preferably exactly one enzyme layer. Even in embodiments in which the test strip has two substrate layers, preferably only one enzyme layer is provided. In such embodiments, it is advantageous if the enzyme layer is arranged above the substrate layers so that the sample applied to the test strip first passes through the enzyme layer and only then diffuses into the substrate layers arranged alongside each other below the enzyme layer. As a result, mixing of the resulting analysis solutions may be prevented.

The test strip according to the invention may contain additional layers in addition to the enzyme layer and the substrate layer. The test strip preferably contains a coating layer onto which the sample is applied. Optionally, a release layer may be provided below the coating layer, in particular which retains cellular components before the sample diffuses into the enzyme and substrate layers. This is particularly advantageous when the sample is a blood sample. The multi-layered structure of the test strip may be held together by one or more adhesive layers and/or by a sheath surrounding the other layers. In preferred embodiments, such a shell may also function as a coating layer. The test strip according to the invention may additionally comprise a carrier layer on which the rest of the layer composite is applied. Such a carrier layer facilitates the handling of the test strip, since it makes it possible to manipulate the test strip, without having to touch the remaining layer structure with its, in part, sensitive layers.

The invention also provides a test system comprising a test strip according to the invention and a detector for detecting the signals S1 and S2. Preferably, the detector is a photometer, more preferably a battery-powered photometer. The term photometer also includes fluorescence-measuring devices. The test system may further include a computing unit for calculating the conversion factor from the detected signals and for determining the concentration of the analyte in the sample. Furthermore, the test system may include an output unit with the help of which the user can read the determined concentration of the analyte.

In another application according to the invention, in which one or both signals are electrochemical in nature, the detector is an ammeter or voltmeter, preferably an ammeter. The detector may further be equipped with signal amplification components. The test system may further include a computing unit for calculating the conversion factor from the detected signals and for determining the concentration of the analyte in the sample. Furthermore, the test system may include an output unit with the help of which the user can read the determined concentration of the analyte.

The invention also relates to the use of the method according to the invention for determining the content of an analyte in a sample.

According to the invention, there is also a kit which is suitable for carrying out the method according to the invention. The kit comprises at least one enzyme preparation and at least one preparation of at least one signal-producing substrate. In this case, the enzyme preparation comprises the above-described enzyme and the preparation of the signal-producing substrate comprises the above-described signal-producing substrate.

The enzyme preparation and/or the at least one preparation of at least one signal-producing substrate may be a solid preparation, for example a lyophilizate or a powder, or a preparation in liquid form, for example a solution. Liquid preparations may comprise solutions in suitable buffers or deionized and/or sterile water. These and other suitable preparations and their preparation are known to those skilled in the art.

The kit preferably provides instructions for carrying out the method according to the invention.

The at least one preparation of at least one signal-producing substrate may preferably contain two or more different signal-producing substrates. These different signal-producing substrates lead to distinguishable signals upon enzymatic conversion.

The kit preferably comprises two preparations of at least one signal-producing substrate, wherein the preparations may each contain the at least one signal-producing substrate in different concentrations. The concentration of the signal-producing substrate in the first preparation may preferably be at least twice as high as the concentration of the signal-producing substrate in the second preparation.

Further preferably, the kit may comprise two preparations of at least one signal-producing substrate, wherein the signal-producing substrate is different in the two preparations.

Furthermore, the kit may preferably comprise at least one cuvette and/or at least one microtiter plate. The measurements to be carried out when using the kit or when implementing the method according to the invention may be carried out using standard laboratory equipment such as fluorescence spectrometers or microtiter plate readers. Appropriate measuring methods are known to the person skilled in the art.

Preferably, the kit provides each of the preparations referred to in separate containers. The containers are preferably closable, in particular closable cuvettes and/or microtiter plates.

### EXAMPLES

In the following, the invention will be illustrated with reference to some embodiments.

Competition BAPNA/Fibrinogen for Cleavage by Trypsin

In a first series of experiments, the competition of BAPNA (N-benzoyl-D,L-arginine-p-nitroanilide) and fibrinogen for cleavage by the enzyme trypsin was investigated. Cleavage of BAPNA by trypsin results in the release of p-nitroaniline and thereby an increase in absorbance at 405 nm.

Influence of Enzyme Activity

First, the enzyme activity was varied to simulate a decrease in enzyme activity during storage of the assay or inhibition of the enzyme by factors contained in the sample. One series of measurements was carried out at an enzyme concentration of 60 μg trypsin per ml of the analysis solution, while the trypsin concentration in the other analysis solution was only 40 μg/ml. In addition, two different BAPNA concentrations (0.2 mM and 2 mM) were tested. The fibrinogen concentration was varied between 0 mg/ml and 4 mg/ml with intermediate values of 1 mg/ml, 2 mg/ml and 3 mg/ml. The measurements were carried out at 25° C.

As the fibrinogen concentration increases, the measured rate of BAPNA conversion decreases. At low BAPNA concentration this effect is relatively stronger. At a saturating concentration, vₘₐₓ is measured independently of the fibrinogen. Increasing the enzyme concentration results in a proportional increase in conversion rate at all concentrations of the signal-producing substrate and analyte. A decrease in enzyme activity during storage of the assay or inhibition by the sample would accordingly lead to greatly increased fibrinogen determination as a result of the decreased activity. This result indicates that an enzyme-kinetic determination of the concentration of the analyte in a sample is influenced sensitively by a decrease in the enzyme activity during storage of the assay or an inhibition of the enzyme by factors contained in the sample, so that a test such as an equivalent one known from the prior art is not suitable for determining the fibrinogen concentration with high accuracy.

Influence of the Measuring Wavelength

Another series of measurements was carried out at a trypsin concentration of 40 μg/ml under the conditions described above, except that measurement was performed at a wavelength of 425 nm instead of the above-mentioned 405 nm.

Due to the difference in the wavelength, significantly reduced values were measured, which resulted in a significant overestimation of the fibrinogen concentration.

Influence of Temperature

Another series of measurements was carried out at a trypsin concentration of 40 μg/ml under the above-described conditions, except that the temperature was 29° C. instead of the above-mentioned 25° C.

The increase in temperature resulted in an increase in enzyme activity of approximately 20%. As a result, such an increase in temperature would falsely indicate a lower fibrinogen concentration.

Influence of Temperature and Enzyme Activity on the Conversion Factor.

Using the competition of BAPNA and fibrinogen for conversion by trypsin as an example, it could be shown that the influence of large differences in enzyme concentration, measuring wavelength and temperature can be eliminated by calculating a conversion factor from the signals of two measurements with different concentrations of the signal-producing substrate.

FIGS. 1 and 2 show the principle using the example of different substrate and enzyme concentrations. FIG. 2 shows that both substrate and enzyme concentrations influence the fibrinogen-dependent conversion rate.

If one divides the conversion rate at high substrate concentration by that at lower concentration, one obtains conversion factors that depend only on the fibrinogen concentration (FIG. 3).

Theoretical Justification for Competitive Interaction

If the analyte and the signal-producing substrate enter a rapid competitive equilibrium with the enzyme and we observe the initial rate of conversion of the substrate as a signal, then we can disregard the conversion of the analyte. Then we can treat the analyte as an inhibitor and use the known Equation 1 for competitive inhibition. If one then plots the normalized conversion rate V/Vₘₐₓ against the normalized substrate concentration [S]/K_{M} (logarithmic scale), different curves result that depend on the normalized analyte concentration [I]/K_{I}, which allow a differentiation of the analyte concentration. This is shown in FIG. 4.

For the high substrate concentration, one preferably chooses such a high substrate concentration that close to vₘₐₓ is achieved, almost independently of the analyte. In the example this works very well at [S]/K_{M}=1000 (FIG. 4, top right rectangle). With lower concentrations (e.g. 50·K_{M}), one obtains a correspondingly higher variation in initial rate values but still a useful reference value for calculating conversion factors.

With the lower substrate concentration, one can shift the differentiation of the analyte concentrations slightly more to the higher values (FIG. 4, center rectangle) or to the lower values (FIG. 4, left rectangle). Very high conversion factors are then obtained (FIG. 5), but account must be taken of the fact that the rate of conversion at the low substrate concentration will be very low and correspondingly difficult to measure. Therefore, low substrate concentrations between 1·K_{M} and 20·K_{M} are preferred.

The quality of differentiation also depends on the normalized analyte concentration [I]/Kᵢ. For a good differentiation [I]≥K_{I} is advantageous. For many enzymes, K_{I} is dependent of the test conditions, e.g. pH or ionic strength, and may be optimized accordingly. It is also possible to carry out an appropriate sample dilution or concentration or, finally, to search for or generate an enzyme with suitable K_{I}.

There are many other types of inhibition such as non-competitive, uncompetitive, mixed or even irreversible. In some cases, such characterizations apply only within certain concentration ranges of inhibitor and substrate. In the case of strongly binding inhibitors, the requirement for reversible pre-equilibrium is also damaged by too slow dissociation of the inhibitor; competitive binding then results in non-competitive inhibition (J. Hones, Habilitation Thesis, University of Stuttgart 1985, p. 59-61).

Therefore, it must be emphasized that the inventive principle is not limited to the above-described theoretical case of competitive inhibition. Regardless of the nature of the enzyme-kinetically determined inhibition, it is always applicable when substrate- and analyte-conversion influence each other. In such cases, a relationship between two measurements at low and high substrate concentrations can eliminate variable enzyme activity. One then works with purely empirical calibration curves of conversion factor versus analyte concentration.

Example Thrombin/Fibrinogen/Fluorescence Measurement

Buffer, substrate and fibrinogen were placed in a 96-well microtiter plate. The enzyme was injected with the injector attached to the TECAN reader. The experimental conditions are set out in the following information.
Substrate conc. Z-Gly-Pro-Arg-aminomethylcoumarin 10 mM in DMSO
Substrate dil. Z-Gly-Pro-Arg-aminomethylcoumarin 1 mM in DMSO
Test buffer 50 mM Tris, 250 mM NaCl, 0.1% PEG6000, pH 8.0
Haemocomplettan 100 mg/ml dist. water≅44 mg/ml of fibrinogen
Enzyme Solution 1 U/ml in 10 mM HEPES, 0.1% bovine serum albumin, pH 6.4
Temperature 30 C

Pipetting Scheme

Pipetting Scheme

| | Well No. | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 11 | 12 | | |
| Substrate conc. | 5 | / | 5 | / | 5 | / | 5 | / | 5 | / | 5 | / | | |
| Substrate dil. | / | 5 | / | 5 | / | 5 | / | 5 | / | 5 | / | 5 | | |
| Test buffer | 85 | 85 | 75 | 75 | 80 | 80 | 83 | 83 | 84 | 84 | 85 | 85 | {close oversize brace} | V in μl |
| Haemocomplettan | 0 | 0 | 10 | 10 | 5 | 5 | 2 | 2 | 1 | 1 | 0 | 0 | | |

| | |
|---|---|
| Enzyme Solution | injection in each case 10 μl for starting the reaction |

After mixing briefly by shaking, the increase in fluorescence by substrate cleavage was recorded in each case for 5 minutes. The Δl/min values were each divided in pairs, i.e. ½, ¾, to 11/12. It was noticeable that the enzyme activity decreased from 100% to 15% within one hour from Well 1 to Well 11. However, there was only a 5-minute time difference between the offset pairs of values and there was a reasonable dependence of the conversion factors on the fibrinogen concentration. Thus, the method is able to compensate for even large errors in the enzyme activities, provided that the differences between the offset compartments are small. The results are shown in FIG. 6.

The instability of the diluted enzyme was due to adsorption on the glass surface of the injector syringe. Addition of 0.1% PEG6000 and 0.25M NaCl stabilized the enzyme.

In fluorescence measurements, the dependence of the fluorescence intensity on the presence of quenching substances (quenchers) is often problematic. Since in the embodiment according to the invention the fluorophore is influenced identically in both compartments, a potential quenching effect is compensated for by the offsetting.

Phenylendiamine Cleavage/Photometric and Electrochemical Detection

Thrombin also cleaves Z-Gly-Pro-Arg-p-phenylenediamine. The product could be detected by oxidative coupling with a-naphthol in the presence of hexacyanoferrate III. The result was a violet dye with Amax=565 nm. The Michaelis-Menten constant at pH 8 (buffer as for fluorescence measurement+2% Triton X100) was 6 μM.

Alternatively, the phenylenediamine may also be electrochemically oxidized and the increase in current over time used as a measure of enzyme activity. With high and lower substrate concentrations, fibrinogen may be measured similarly as by fluorescence. Electrochemical measurements map the substance transport to the electrode surface. This is proportional to the concentration and the diffusion coefficient. Especially in biological sample material this is often very variable, e.g. due to macromolecules or even blood cells/hematocrit. Because of this, there is, for example when used for blood glucose tests, a wealth of additional measurements, e.g. complex impedance, which detect and correct these variations. In the method according to the invention, the viscosity and the hematocrit in both compartments are the same and are compensated for by the offsetting.

### BRIEF DESCRIPTION OF THE FIGURES

FIG. 1 shows the course of the function U([I] for [S₂]=K_{M} and [S₁]=10*K_{M}.

FIG. 2 shows the rate of conversion of BAPNA (y-axis) as a function of the fibrinogen concentration (x-axis). Shown are four different series of measurements, wherein both the concentration of the signal-producing substrate and the concentration of the enzyme were varied.

FIG. 3 shows the dependence of the conversion factor calculated from the data shown in FIG. 2 on the fibrinogen concentration. The conversion factors at two different enzyme concentrations as well as the calculated regression line are shown.

FIG. 4 shows the normalized conversion rate V/Vₘₐₓ as a function of the normalized substrate concentration [S]/K_{M} (logarithmic scale). The dependency for various analyte concentrations is shown.

FIG. 5 shows the dependence of the conversion factor (y-axis) on the normalized analyte concentration [I]/K_{I}. The dependency for various substrate concentrations is shown.

FIG. 6 shows the conversion factor (y-axis) as a function of the fibrinogen concentration (x-axis). The numbers 1 to 6 refer to the series of measurements shown in the pipetting scheme, where “1” in FIG. 6 corresponds to the offsetting of measurement series 1 and 2, “2” to measurement series 3 and 4, “3” to measurement series 5 and 6, “4” to measurement series 7 and 8, “5” to measurement series 9 and 10, and “6” to measurement series 11 and 12.

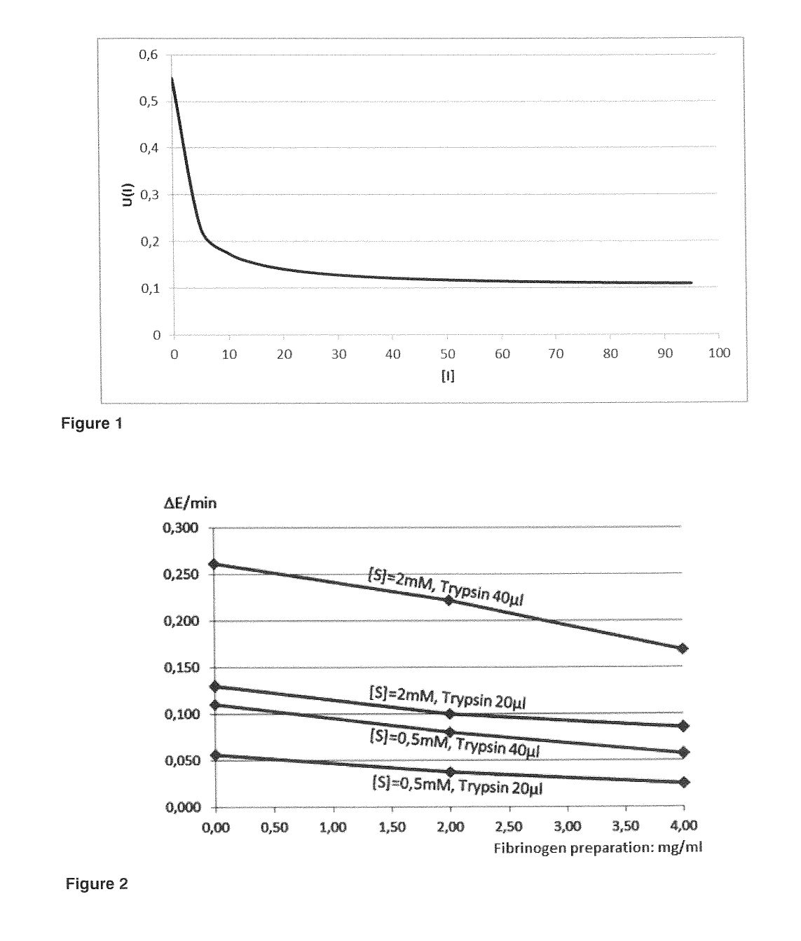

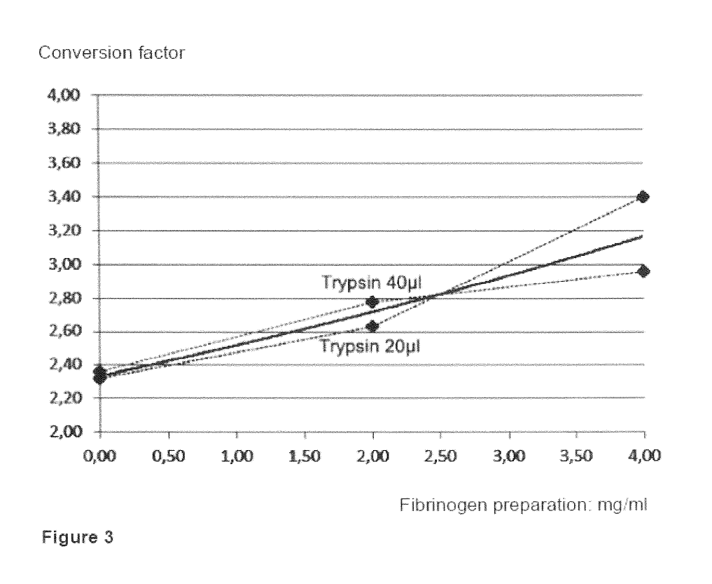

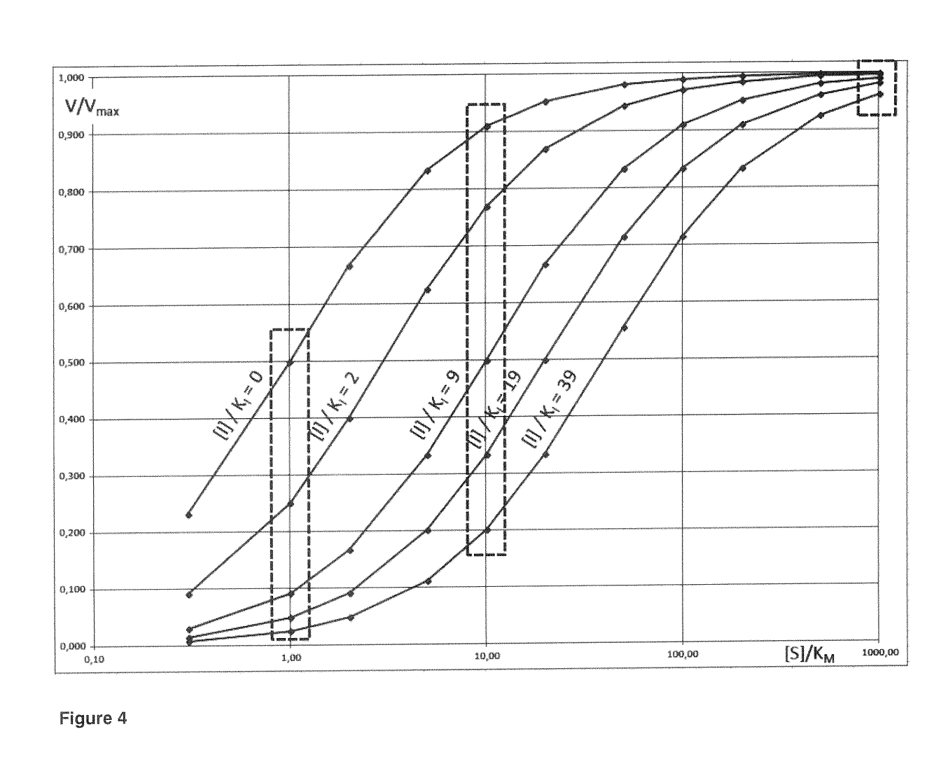

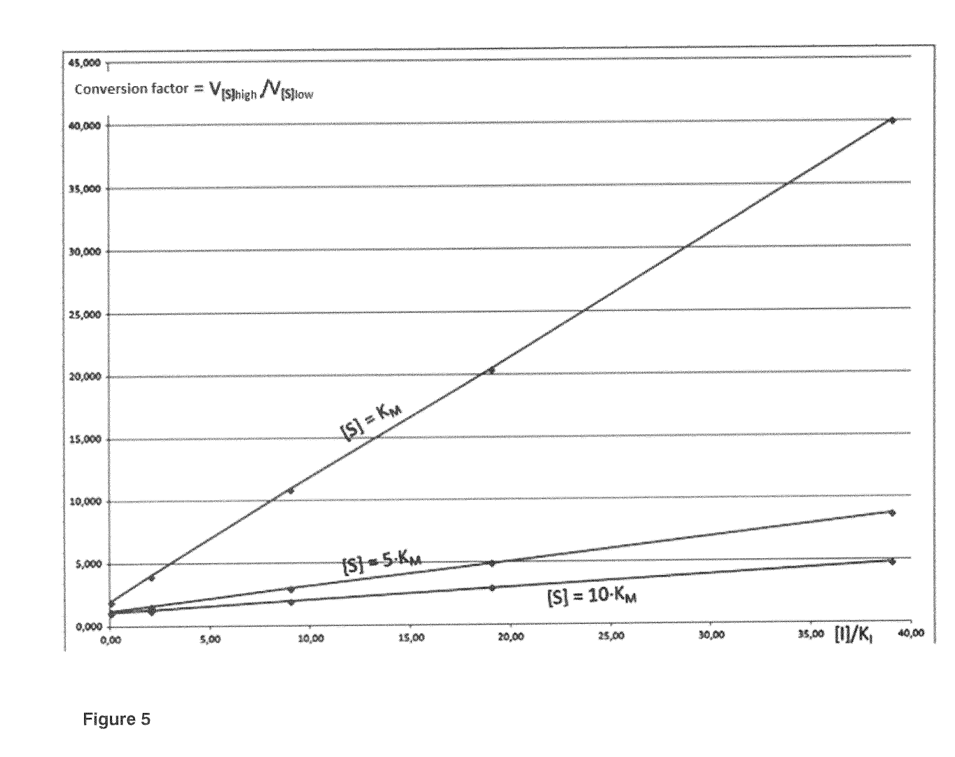

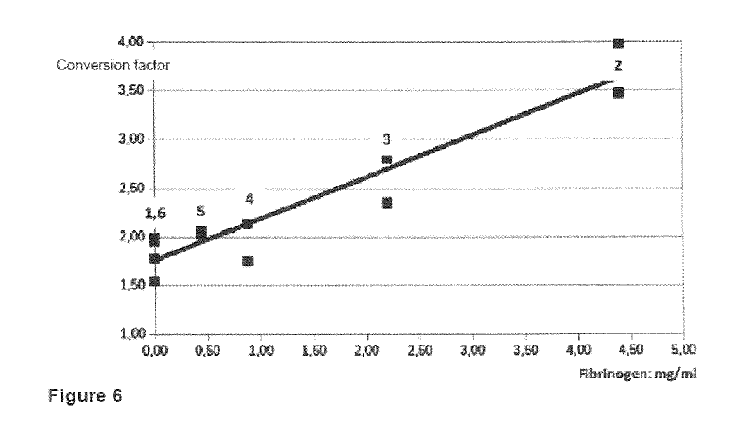

What is claimed is:

1. A method for determining the level of an analyte in a sample comprising the following steps:
a. providing at least one analysis solution,
i. wherein the at least one analysis solution comprises in each case an enzyme, a signal-producing substrate and a known proportion of the sample to be analyzed, and
ii. wherein the enzyme is capable of converting both the analyte and the signal-producing substrate so that the analyte and signal-producing substrate compete for conversion by the enzyme,
b. detection of two signals S1 and S2 produced by enzyme-catalyzed conversion in the at least one analysis solution,
c. calculation of a conversion factor from the signals, and
d. determination of the content of the analyte in the sample by means of the conversion factor.

2. The method according to claim 1, wherein in step a, a first analysis solution A1 and a second analysis solution A2 are provided.

3. The method according to claim 2, wherein the concentration of the signal-producing substrate in the first analysis solution A1 is at least twice as high as the concentration of the signal-producing substrate in the second analysis solution A2.

4. The method according to claim 2, wherein the concentration of the signal-producing substrate in the first analysis solution A1 is at least 80% of the concentration required for saturation of the enzyme.

5. The method according to claim 1, wherein the conversion factor is calculated from the signals by determining the initial rates v₀(S1) and v₀(S2) of the enzymatic conversion of the signal generating substrate from the signals and these initial rates v₀(S1) and v₀(S2) are offset with each other.

6. The method according to claim 5, wherein the offsetting of the initial rates includes forming a quotient of v₀(S1) and v₀(S2).

7. The method according to claim 1, wherein the enzyme is thrombin and the analyte is fibrinogen.

8. The method according to claim 1, wherein the signals are produced through:
a. enzyme-catalyzed conversion of a higher concentration of the signal-producing substrate (signal S1) and by enzyme-catalyzed conversion of a lower concentration of the signal-producing substrate (signal S2) in two separate analysis solutions, or
b. by enzyme-catalyzed conversion of the signal-producing substrate (signal S1) and by enzyme-catalyzed conversion of the analyte (signal S2), or
c. enzyme-catalyzed conversion of a first signal-producing substrate (signal S1) and enzyme-catalyzed conversion of a second signal-producing substrate (signal S2).

9. The method according to claim 1, wherein the analysis solution is provided in cuvettes and/or microtiter plates.

10. A test strip for performing the method according to claim 1, wherein the test strip comprises at least one enzyme layer containing the enzyme and at least one substrate layer containing the signal-producing substrate.

11. The test system comprising a test strip according to claim 9 and a detector for detecting the signals S1 and S2.

12. A kit for carrying out the method according to claim 1, wherein the kit comprises at least one enzyme preparation and at least one preparation of at least one signal-producing substrate.

13. A kit comprising at least one enzyme preparation and at least one preparation of at least one signal-producing substrate, further comprising instructions for carrying out the method according to claim 1.

14. The kit according to claim 12, wherein the kit comprises two preparations of at least one signal-producing substrate, wherein each of the preparations contain the at least one signal-producing substrate in different concentrations.

15. The kit according to claim 12, wherein the kit comprises two preparations of at least one signal-producing substrate, wherein the signal-producing substrate is different in the two preparations.

16. The kit according to claim 12, wherein the at least one preparation contains at least two different signal-producing substrates.

17. The kit according to claim 14, wherein the concentration of the signal-producing substrate in a first preparation is at least twice as high as the concentration of the signal-producing substrate in a second preparation.

18. The kit according to claim 12, wherein the kit further comprises at least one cuvette and/or at least one microtiter plate.

19. The kit according to claim 12, wherein the enzyme preparation and the preparation of the at least one signal-producing substrate are provided in separate containers.
